(19) United States
(12) Patent Application Publication
(10) Pub. No.: US 2011/0086017 A1
(43) Pub. Date: Apr. 14, 2011

(54) Medical Food composition and methods for management of inflammatory processes in mammals

(76) Inventors: Svetlana Kravets, Granada Hills, CA (US); Alexandre Kravets, Granada Hills, CA (US); Matthew Jacobson, Santa Monica, CA (US)

(21) Appl. No.: 12/885,530

(22) Filed: Sep. 19, 2010

Related U.S. Application Data
(60) Provisional application No. 61/249,998, filed on Oct. 8, 2009.

(51) Int. Cl. A61K 38/48; A61P 29/00; A61P 19/02

(52) U.S. Cl. 424/94.63

(57) ABSTRACT

A medical food composition, containing at least source of milk protein derived from milk producing animals, exposed to immune stimulants during pregnancy and lactation period, source of Curcuminoids, source of proteolytic enzymes, and source of Piperin effective to manage inflammatory response and associated pain symptoms in mammals.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The present invention is in the field of nutrition. More particularly, the present invention provides a medical food composition and methods effective to manage inflammatory response in mammals.

Inflammation, as defined in Dorland's Medical Dictionary, is “a localized protective response elicited by injury or destruction of tissues which serves to destroy, dilute or wall off (sequester) both the injurious agent and the injured tissue. The classical signs of acute inflammation are pain (dolor), heat (calor), redness (rubor), swelling (tumor), and loss of function (functio laesa).”

The inflammatory response is any response characterized by inflammation as defined above. It is well-known to those skilled in the medical arts that while the inflammatory response is a natural response of the body to injury or disease, it causes much of the physical discomfort, i.e., pain and loss of function. Accordingly, it is a common medical practice to administer pharmacological agents which have the effect of neutralizing the inflammatory response. Agents having these properties are classified as anti-inflammatory drugs.

Anti-inflammatory drugs are used for the treatment of a wide spectrum of disorders and the same drugs are often used to treat different diseases. Treatment with anti-inflammatory drugs is not for the disease but rather for the symptom, i.e., inflammation.

Non-steroidal anti-inflammatory drugs (NSAIDs) represent the most widely used class of compounds for the treatment of the inflammatory response.

NSAIDs are assumed to be well tolerated and are widely used as an initial therapy for common inflammation. They range from over the counter Aspirin and Ibuprofen to a host of prescription brands. These pharmaceutical agents constitute one of the most widely used class of drugs, with more than 70 million prescriptions and more than 30 billion over-the-counter tablets sold annually in the United States alone.

While NSAIDs are widely used in medical practice, recent studies present some alarming statistics concerning the safety of these drugs. In July of 1998, the American Journal of Medicine reported:

“Conservative calculations estimate that approximately 107,000 patients are hospitalized annually for non-steroidal anti-inflammatory drug (NSAID)-related gastrointestinal (GI) complications and at least 16,500 NSAID-related deaths occur each year among arthritis patients alone. The figures of all NSAID users would be overwhelming, yet the scope of this problem is generally under-appreciated.”

In addition, NSAID-related gastrointestinal side effects account for one third of the cost for arthritis therapy.

A new study published in the January 26, 2009, issue of the Archives of Internal Medicine, reported further evidence that even commonly used NSAIDs are harmful to heart-failure patients. The study reported dose-related increases in risk of death and re-hospitalization for heart failure or Myocardial Infarction with all COX-2 inhibitors or other NSAIDs. (Gislason G H, Rasmussen J N, Abildstrom S Z, et al. Increased mortality and cardiovascular morbidity associated with use of nonsteroidal anti-inflammatory drugs in chronic heart failure. Arch Intern Med 2009; 169:141-149.)

Accordingly, in spite of the large number of anti-inflammatory agents that are currently available, there is a need for a safe, effective, anti-inflammatory product which is free of severe side effects and adverse reactions associated with NSAIDs.

A natural, easily administrable and readily available food product with anti-inflammatory effects comparable to those of NSAIDs would be a safe therapeutic composition for those wishing to avoid the risk of side effects associated with NSAIDs.

It has been known in the prior art to produce food products having a variety of therapeutic effects. For example, U.S. Pat. Nos. 4,284,623, 4,956,349, 5,194,255, 5,242,691, 5,352,462, 5,650,175, and 5,980,953 disclose methods and processes related to the extraction and use of Hyperimmune milk protein concentrate, containing anti-inflammatory peptides.

It is also known to those skilled in the art of nutrition that nutritional products containing Curcuminoids and proteolytic enzymes have a positive anti-inflammatory effect when the administered dosage is sufficient to produce such an effect.

A combination of such ingredients in amounts sufficient to produce desirable effect would result in a product that is comparable in its efficacy with most NSAIDs, but at the same time is free of their side effects.

It is also known to those skilled in the art of nutrition that the effective daily dosage of such a natural source of proteolytic enzymes as Bromelain is approximately 320 mg a day. This is the dosage administered to post-surgical patients in German hospitals where Bromelain is used for managing post-surgical inflammation.

It is also known to those skilled in the art of nutrition that the effective dosage of Curcumin ranges from 400 mg per day to 1200 mg per day (Chandra D, Gupta S. Anti-inflammatory and anti-arthritic activity of volatile oil of Curcuma longa (Haldi). Ind J Med Res 1972;60:138-142; Arora R, Basu N, Kapoor V, et al. Anti-inflammatory studies on Curcuma longa (turmeric). Ind J Med Res 1971;59:1289-1295; Srivastava R. Inhibition of neutrophil response by Curcumin. Agents Actions 1989;28:298-303).

While all the ingredients required to produce a natural food product comparable in its efficacy to prescription NSAIDs are readily available, the amount of the substance required to produce such an effect presents certain challenges in manufacturing and administrability of the compounded product. This is due to the low density of components and the required dosage to produce a positive therapeutic effect, which either prevents encapsulation of a sufficient amount into a standard size capsule, or impairs administrability of the product by requiring an unreasonably large number of standard sized capsules to be taken per day to produce the desirable effect.

It has been known in the prior art to produce food products to improve gastrointestinal absorption and systemic utilization of nutrients. For example, U.S. Pat. Nos. 5,744,161 and 5,972,382 describe the mechanism of action and the process of extraction and purification of Piperin. Piperin is a pungent substance found in plants of the Piperaceae family—including Piper Nigrum (black pepper) and Piper Longum (long pepper).

Addition of Piperin to herbal, pharmaceutical and food products increases their bioavailability, allowing for administration of smaller dosage to achieve desirable effect or for acceleration in achieving such effect.

### SUMMARY OF THE INVENTION

The present invention provides a medical food composition and methods effective to manage inflammatory response and related symptoms in mammals.

The present invention is directed to all-natural preparations and methods of mass producing and using such preparations to alleviate some, preferably all symptoms of inflammatory response in mammals.

The medical food composition of the present invention, preferably encapsulated or compressed powder, is comprised of the following mixture:
concentrated milk protein derived from milk producing animals, exposed to immune stimulants during pregnancy and lactation period (hyperimmune milk protein concentrate);
source of Proteolytic enzymes;
source of Curcuminoids;
source of Piperin;

Other nutritional or herbal ingredients, scientifically proven to further benefit mitigation of inflammatory response or underlying condition, may be added to this composition.

The methods of the present invention include but are not limited to:
Accelerating and increasing therapeutic effect of anti-inflammatory components of the present invention by increasing their bioavailability, achieved through addition of Piperin source to the composition of the present invention;
Administering to a mammal, suffering from the effects of the inflammatory response, an effective amount of the medical food of the present invention

### DETAILED DESCRIPTION OF THE INVENTION

The subject of this patent application is the medical food composition and methods, intended for use in nutritional management of inflammatory processes and related pain symptoms. By way of a non-limiting example, the term “inflammatory processes” includes effects of such trauma- or chronic-condition-related inflammation of soft tissues and/or joints as arthritis, bursitis, strains and sprains of muscles, asthma, and post-surgical inflammation.

The term “medical food,” as defined in section 5(b) of the Orphan Drug Act (21 U.S.C. 360ee (b) (3)) is “a food which is formulated to be consumed or administered enterally under the supervision of a physician and which is intended for the specific dietary management of a disease or condition for which distinctive nutritional requirements, based on recognized scientific principles, are established by medical evaluation.”

This definition separates medical foods from nutritional supplements and other food products and is pivotal to the methods of the present invention due to the medical supervision requirement. This is because Piperin, a component of the present invention, has a high drug interaction rate due to its unique ability to increase bioavailability of certain drug agents.

The composition of the present invention includes:
concentrated milk protein derived from milk producing animals, exposed to immune stimulants during pregnancy and lactation period, also known as hyperimmune milk protein concentrate and described in previously referenced U.S. Pat. Nos. 4,284,623, 4,956,349, 5,194,255, 5,242,691, 5,352,462, 5,650,175, and 5,980,953 in the amount ranging from 1,000 mg to 4,000 mg;
source of Curcuminoids, which by the way of non-exclusive example, may be in a form of a turmeric plant extract, in the amount ranging from 200 mg to 1,200 mg depending on the Curcuminoids content;
source of proteolytic enzymes, which by the way of non-exclusive example may be in a form of Bromelain, in the amount ranging from 200 mg to 750 mg;
source of Piperin in the amount ranging from 5 mg to 20 mg;

Other nutritional or herbal ingredients, scientifically proven to further benefit mitigation of inflammatory response or underlying condition, may be added to this composition.

In 1955, Petersen and Campbell presented evidence that cow's milk, immunized with a polyvalent vaccine made from human pathogenic bacteria, contained high levels of antigen-specific antibodies. (Petersen W E, Campbell B. Use of Protective Principles in Milk and Colostrum in Prevention of Disease in Man and Animals. Lancet. 1955;75:494)

Since 1958, research conducted on the health benefits of hyperimmune milk from cows exposed to a specific proprietary immune stimulant indicates that, among other benefits, it has been shown to have anti-inflammatory qualities. Hyperimmune milk is derived from a process that involves the delivery of an immune stimulant to pregnant cows, beginning 4 weeks before parturition and continuing every 2 weeks throughout lactation. Exposing cows to a proprietary immune stimulant results in an increase in the synthesis and excretion, in the milk, of anti-inflammatory components that are normally present in cow's milk. (The Ohio Survey Results and Methods, 1961-1989, Archives. In: Stolle J R, Beck L R. Stolle Immune Milk. Cincinnati, Ohio: Stole Milk Biologics, Inc. [SMBI]; Cincinnati, Ohio, 1989:32-55.)

The concentrated formulation of the hyperimmune milk powder derived from this process, MPC, was used in the study to test anti-inflammatory activity in adults. This study showed that MPC resulted in the increased expression of naturally occurring, biologically active factors in the milk. In addition to high-molecular-weight (HMW) immunoglobulins (Ig) (antigen-specific IgG antibodies), the milk also was discovered to have low-molecular-weight (LMW) components with anti-inflammatory activity. (Ormrod D J, Miller T E. The Anti-Inflammatory Activity of a Low Molecular Weight Component Derived from the Milk of Hyperimmunized Cows. Agents & Actions. 1991;32:160-166)

Multiple mechanisms are involved in the suppression of inflammation by glucocorticoids, including decreased release of vasoactive and chemoattractive factors, and decreased emigration of neutrophils to areas of injury and inflammation. Miller et al. have shown activity similar to that of glucocorticoids involving neutrophil activity and emigration with hyperimmune milk-derived anti-inflammatory compounds. (Ormrod D J, Miller T E. A Low Molecular Weight Component Derived from the Milk of Hyperimmunized Cows Suppresses Inflammation by Inhibiting Neutrophil Emigration. Agents & Actions. 1992;35:1-10)

Supplementing the diet with naturally occurring, biologically active factors found in the hyperimmune milk protein concentrate has shown to inhibit inflammation by decreasing the emigration of neutrophils and may do so by restricting extravasation of neutrophils through vascular tight junctions. The results with nutritional supplementation with hyperimmune milk protein concentrate revealed a significant improvement in joint pain, stiffness, and swelling in the treatment group compared with the control group (P<0.015). Parallels can be drawn between the activities of other anti-inflammatory agents such as Glucocorticoids (e.g. cortisol) that share similar anti-inflammatory properties linked to the inhibition of neutrophil functions. These very unique anti-inflammatory mechanisms of action explain the significant improvements in joint pain, stiffness and immobility observed in the clinical study and also account for the favorable side effect profile in the study group. (Zenk J L, Helmer T R, Kuskowski M A. The Effects of Milk Protein Concentrate on the Symptoms of Osteoarthritis in Adults: An Exploratory, Randomized, Double-Blind, Placebo-Controlled Trial. Curr Ther Res, 2002;63:430-442.)

Curcuminoids are nutrients that have been shown to possess unique antioxidant and anti-inflammatory properties. The anti-inflammatory strength of Curcuminoids is comparable to steroidal drugs such as indomethacin. (Srivastava V, et al. Effect of Curcumin on Platelet Aggregation and Vascular Prostacyclin Synthesis. Arzneim Forsch/Drug Res. 1986;36:715-17.)

Curcuminoids reportedly inhibit enzymes, which participate in the synthesis of inflammatory substances (leukotrienes and prostaglandins) derived from arachidonic acid, and it is claimed they are comparable in activity to the NSAID. In a double-blind study of individuals with rheumatoid arthritis, Curcuminoid products produced significant improvement in all subjects (Deodhar S D, et al. Preliminary Studies on Anti-Rheumatic Activity of Curcumin. Ind J Med Res. 1980;71:632; Ammon H P, et al. Mechanism of Anti-inflammatory Actions of Curcumin and Boswellic Acids. J Ethnopharmacol. 1993;38:113.)

Curcuminoids reportedly have a similar action to that of aspirin, aspirin-like anti-inflammatory agents and Cox-2 inhibitors (Srivastava K C, et al. Curcumin, A Major Component of Food Spice Turmeric (Curcuma longa) Inhibits Aggregation and Alters Eicosanoid Metabolism In Human Blood Platelets. Prostaglandins Leukot Essent Fatty Acids. Apr 1995;52(4):223-27.) However, Curcuminoid products are superior to aspirin, since Curcuminoids, unlike aspirin, are reported to selectively inhibit synthesis of inflammatory prostaglandins but do not affect the synthesis of prostacyclin (Srivastava V, et al. Effect of Curcumin on Platelet Aggregation and Vascular Prostacyclin Synthesis. Arzneim Forsch/Drug Res. 1986;36:715-17). Curcuminoids may be preferable for individuals who are prone to vascular thrombosis and require anti-inflammatory and/or anti-arthritic therapy.

Many studies have confirmed the effectiveness of anti-inflammatory properties possessed by Curcuminoids. Study by Youn H S, Saitoh S I, Miyake K, Hwang D H (Youn H S, Saitoh S I, Miyake K, Hwang D H. Inhibition of homodimerization of Toll-like receptor 4 by Curcumin. Biochem Pharmacol. 2006 Jun 28;72(1):62-9. Epub 2006 Apr 1) proved that Curcumin has been shown to decrease the activation of NF-kappaB induced by multiple inflammatory stimuli by preventing IKKbeta kinase activity in MyD88-dependent pathway. Additionally, Curcumin prevented LPS-induced IRF3 activation. This study reported that Curcumin inhibits ligand-induced and ligand-independent dimerization of TLR4. This all shows that pytochemicals such as Curcumin can modulate immune and inflammatory responses.

Results of the study published in Critical Care Medicine show that Curcumin is effective in managing even septic conditions. (Siddiqui A M, Cui X, Wu R, Dong W, Zhou M, Hu M, Simms H H, Wang P. The anti-inflammatory effect of Curcumin in an experimental model of sepsis is mediated by up-regulation of peroxisome proliferator-activated receptor-gamma. Crit Care Med. 2006 Jul;34(7):1874-82)

Proteolytic enzymes are nutrients that have displayed anti-inflammatory and analgesic properties in human and laboratory studies. One of the sources of proteolytic enzymes is Bromelain. Bromelain is a proteolytic enzyme found in pineapple. (Brien S, Lewith G, Walker A F, et al. Bromelain as an adjunctive treatment for moderate-to-severe osteoarthritis of the knee: a randomized placebo-controlled pilot study. QJM. 2006 Dec;99(12):841-50; Klein G, Kullich W, Schnitker J, Schwann H. Efficacy and tolerance of an oral enzyme combination in painful osteoarthritis of the hip. A double-blind, randomised study comparing oral enzymes with non-steroidal anti-inflammatory drugs. Clin Exp Rheumatol. 2006 Jan-Feb;24(1):25-30.).
The action of Bromelain is attributed to the inhibition of thromboxane synthesis or bradykinin production. It has been shown to increase fibrinolytic activity and indirectly inhibit pro-inflammatory prostaglandins. Bromelain has been found to inhibit the biosynthesis of pro-inflammatory prostaglandins by lowering kininogen and bradykinin in serum and tissues, and may alter prostaglandin synthesis. It has also been found to activate plasmin production from plasminogen and reduce kinin by inhibiting the conversion of kininogen to kinin. In animal study, Bromelain dose-dependently reduced plasma exudate at the site of inflammation by decreasing bradykinin levels and pre-kallikrein levels. (Majima M, Kawashima N, Hiroshi I, Katori M. Effects of an orally active non-peptide bradykinin B2 receptor antagonist, FR173657, on plasma exudation in rat carrageenan-induced pleurisy. Br J Pharmacol. 1997;121(4):723-730)

An essential component of the present invention is inclusion of Piperin into the composition of the present invention.

Piperin is a pungent substance found in plants of the Piperaceae family—including Piper nigrum (black pepper) and Piper longum (long pepper). These peppers have been used in Ayurvedic medicine for the treatment of various diseases and discomforts. Recent research has provided support for some of these uses and has uncovered the probable mechanism responsible for them.

The mechanism of action as described in previously referenced U.S. Pat. Nos. 5,744,161 and 5,972,382, is believed to be two-fold: by affecting various active and passive transport mechanisms; and by causing the increase for the substrate demand due to enhanced metabolism at the cellular level.

Piperin is commercially available. For example, Sabinsa Corporation offers Piperin extract under the trade mark of BioPerine®. According to Sabinsa Corporation's report, effects of Piperin have been measured in several clinical studies with healthy volunteers in the U.S. These studies measured the absorption of three distinct categories of products. The categories evaluated with and without BioPerine® were fat-soluble (beta-carotene), water-soluble (vitamin B₆) and a mineral(selenium, in the form of selenomethionine). Gastrointestinal absorption of all the studied nutrients, as measured by amounts present in the blood, increased dramatically when administered with BioPerine® as compared to the control group receiving the nutrient alone. Selenium levels increased by 30%, beta-carotene increased by 60%, and the vitamin B₆ increase was slightly higher than beta-carotene (http://www.bioperine.com/clinical.html).

Sabinsa Corporation also conducted a special study of effects of BioPerine® on absorption of Curcuminoids. The result of this study revealed an over 50% increase in relative bioavailability of Curcuminoids when administered with BioPerine® (http://www.bioperine.com/curcumin.html).

The advantages of the present invention include, without limitation, that the composition of the present invention allows for preservation of multi-targeted anti-inflammatory effect of the composition while reducing the dosage necessary to produce such effect. This allows for mass production of standard size 00 capsules containing the composition of the present invention without impairing its administrability which remains on a generally acceptable level of 2 capsules 2 to 4 times daily. The present invention also provides methods for accelerated reduction of inflammatory processes and related pain symptoms in acute stages due to increased bioavailability of its therapeutic components.

### EXAMPLE

A medical food is provided under medical supervision in two servings per day as encapsulated powder. The daily dosage formulation comprises MicroLactin® (Powdered Hyperimmunized Milk Protein Concentrate)—2,000 mg; Turmeric (std. 95% Curcuminoids)—300 mg; Bromelain (600 GDU)—250 mg; Bioperine® (Piper Nigrum Extract)—10 mg.
Directions: Under medical supervision, 2 capsules are administered with an 8 oz. glass of water twice daily.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

What is claimed is:

1. Medical Food comprising at least milk protein derived from milk producing animals, exposed to immune stimulants during pregnancy and lactation period, source of Curcuminoids, source of proteolytic enzymes, and source of Piperin.

2. A method for accelerated control of inflammatory response in mammals by administering an effective amount of the medical food of claim 1 to a mammal at least once per day.

3. A method for accelerated alleviation of inflammation-related pain comprising the step of administering an effective amount of the medical food of claim 1 to a mammal at least once per day.

4. A method for providing long-lasting relief from inflammation-related pain comprising the step of administering medical food of claim 1 to a mammal at least once per day.

5. A method for accelerated improvement of joint function comprising the step of administering an effective amount of the medical food of claim 1 to a mammal at least once per day.

6. A method for inducing a rapid reduction in neutrophil infiltration at a site of injury comprising the step of administering an effective amount of the medical food of claim 1 to an affected mammal at least once per day.

7. A method for accelerated reduction of inflammatory processes at a site of injury comprising the step of administering medical food of claim 1 to a mammal at least once per day.

8. The method of claim 1, wherein medical food is administered under medical supervision at least once daily.

9. Medical food of claim 1, wherein said medical food is provided as a capsule, powder, or tablet for oral delivery.

10. Medical food of claim 1, wherein said medical food is mixed with other nutrients or herbal products.
